(19) United States
(12) Patent Application Publication
(10) Pub. No.: US 2022/0054666 A1
(43) Pub. Date: Feb. 24, 2022

(54) VENTILATOR FILTER STERILIZATION SYSTEMS AND METHODS

(71) Applicant: COVIDIEN LP, Mansfield, MA (US)

(72) Inventors: Matthew J. Phillips, Carlsbad, CA (US); Amand Kasimatis, Carlsbad, CA (US); Julio Jenaro, Roscam (IE)

(21) Appl. No.: 17/393,833

(22) Filed: Aug. 4, 2021

Related U.S. Application Data
(60) Provisional application No. 63/069,457, filed on Aug. 24, 2020.

(51) Int. Cl. A61L 2/00; A61L 2/24; A61M 16/10

(52) U.S. Cl. CPC A61L 2/0047; A61L 2/24; A61M 16/1055; A61M 2209/10; A61L 2202/122; A61L 2202/14; A61L 2202/21; A61L 2202/11

(57) ABSTRACT

A filter sterilization system includes an expiratory filter having filter material that collects pathogens present in the exhaled gas stream from a ventilated patient. A filter sterilizer includes an ultraviolet (UV) light source that is activated by the system to emit light towards the expiratory filter. Additionally, the system includes a ventilator coupled to a patient breathing circuit that provides a gas mixture from a gas source to the ventilated patient and transfers exhaled gases of the ventilated patient to the expiratory filter.

### CROSS-REFERENCE TO RELATED APPLICATION

The present application claims priority to and the benefit of U.S. Provisional Application No. 63/069,457, entitled “VENTILATOR FILTER STERILIZATION SYSTEMS AND METHODS” and filed Aug. 24, 2020, the specification of which is hereby incorporated by reference in its entirety for all purposes.

### BACKGROUND

The present disclosure relates generally to medical devices and, more particularly, to systems, devices, and related methods for sterilizing filters, such as expiratory filters, associated with ventilated patients.

This section is intended to introduce the reader to various aspects of art that may be related to the present disclosure, as described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tube or other medical device may be used to control the flow of air or other gases through a patient's trachea. Indeed, a medical provider may couple a ventilator to an exposed end of the tube and utilize the ventilator to mechanically control the type and amount of gases flowing into and out of the patient's airway. The ventilator may include multiple filters, such as an intake filter at a gas intake portion of the ventilator, a heat and moisture exchanger (HME) at the patient's airway, and, in certain cases, an expiratory filter that is part of an expiratory pathway of the ventilator. The intake filter may clean the gas mixture of undesired particles or condensates to facilitate appropriate and beneficial treatment of the patient. The HME may improve ventilation by humidifying and filtering the gas mixture provided to the patient.

Intubated patients may be infected with contagious pathogens that are present in the lungs and in the patient's exhalation stream. The expiratory filter may collect contaminants, pathogens, or residual medications exhaled by the patient, thereby providing a cleaned air stream that is suitable to be released into the ambient air while protecting medical caregivers from exposure and components of the ventilator from contamination or degradation. In some situations, handling or replacing the expiratory filter places additional demands on hospital personnel to wear personal protective equipment to protect themselves against exposure to contagious pathogens collected and retained by the expiratory filter.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In an embodiment, a system is provided that includes a ventilator comprising a housing having an expiratory port, and an expiratory filter located downstream of the expiratory port, wherein the expiratory filter comprises filter material that collects pathogens from expiratory gases received at the expiratory port. Additionally, the system includes a filter sterilizer coupled to the expiratory filter, wherein the filter sterilizer comprises an ultraviolet (UV) light source oriented at the filter material.

In an embodiment, a filter sterilizer is provided that includes a housing sized and shaped to accommodate an expiratory filter; a plurality of ultraviolet (UV) light sources coupled to the housing; and a controller communicatively coupled to the plurality of UV light sources, wherein the controller: receives an indication of a presence of the expiratory filter in the housing; and activates at least one UV light source of the plurality of UV light sources based on the indication.

In an embodiment, a system is provided that includes an expiratory filter that receives exhalation gases from an expiratory limb of a breathing circuit of a ventilated patient, wherein the expiratory filter comprises filter material that collects contaminants present in the exhalation gases; and an ultraviolet (UV) light source oriented at the expiratory filter; and a controller communicatively coupled to the UV light source, wherein the controller: activates the UV light source to emit a UV light dose to the filter material of the expiratory filter; and generates a signal indicative of completed sterilization in response to the UV light dose being emitted.

Features in one aspect or embodiment may be applied as features in any other aspect or embodiment, in any appropriate combination. For example, any one of a system, monitor, ventilator, controller (e.g., processor-based controller), filter sterilizer, or method features may be applied as any one or more other of system, monitor, ventilator, controller, filter sterilizer, or method features.

### BRIEF DESCRIPTION OF THE DRAWINGS

Advantages of the disclosed techniques may become apparent upon reading the following detailed description and upon reference to the drawings in which:

FIG. 1 is a schematic illustration of a ventilated patient and a ventilation system, in accordance with certain embodiments of the disclosure;

FIG. 2 is a block diagram of an implementation of a filter sterilization system for an expiratory filter of a ventilator that may be used in conjunction with the ventilation system of FIG. 1, in accordance with certain embodiments of the disclosure;

FIG. 3 is a schematic illustration of the filter sterilization system of FIG. 2, in accordance with certain embodiments of the disclosure;

FIG. 4 is a flow diagram of a method of operating the filter sterilization system of FIG. 2 to disinfect the expiratory filter, in accordance with certain embodiments of the present disclosure;

FIG. 5 is a schematic illustration of an implementation of the filter sterilizer of FIG. 2 with an expiratory filter, in accordance with certain embodiments of the present disclosure;

FIG. 6 is a schematic illustration of an implementation of the filter sterilization system of FIG. 2 with a filter sterilizer that includes a sensor, in accordance with certain embodiments of the present disclosure;

FIG. 7 is a flow diagram of a method of operating the filter sterilizer system of FIG. 2 based on a type of the expiratory filter, in accordance with certain embodiments of the present disclosure;

FIG. 8 is a schematic illustration of a user interface for the filter sterilization system of FIG. 2, in accordance with certain embodiments of the present disclosure; and

FIG. 9 is a flow diagram of a method of operating the filter sterilizer system of FIG. 2 in response to a rapid sterilization request, in accordance with certain embodiments of the present disclosure.

### DETAILED DESCRIPTION OF SPECIFIC EMBODIMENTS

An expiratory filter may be included as part of a patient breathing circuit, e.g., positioned within or proximate to a ventilator housing at a point in the breathing circuit downstream of the patient. The expiratory filter operates to collect airborne, vapor, or droplet-based contaminants, such as pathogens, moisture, or residual medications, from exhalation gases of a ventilated patient. The expiratory filter may include a filter housing supporting or retaining filter material that filters the exhalation gases coming from the exhalation limb of the breathing circuit. By removing the contaminants present in the exhalation gases and collecting them on or in the filter material, the expiratory filter facilitates the release of filtered, cleansed air into the ambient environment, thereby improving the air quality for all occupants of a room. For environmental protection and ventilator maintenance, the expiratory filter may be changed periodically and/or between patients. However, during filter exchange, a technician may be exposed to contaminants retained in the filter material. Further, the exchange process may disturb the filter material, inadvertently releasing pathogens into the ambient environment. In addition, the disposal of the used filter material may be regulated according to waste disposal guidelines for biohazardous material, which increases the complexity of the exchange and disposal protocol.

With this in mind, filter sterilization systems and methods are provided herein to facilitate efficient sterilization of ventilator filters, such as expiratory filters, via the application of ultraviolet (UV) light. The filter sterilization techniques operate on the expiratory filter while the expiratory filter is in use to sterilize pathogens or contaminants collected in the filter, reducing the risk and complexity of filter exchange and disposal. For example, a filter sterilizer of the filter sterilization system may include a light source, such as a UV light source. The UV light source is oriented to transmit the UV light (e.g., UV-C germicidal light) into filter material of the expiratory filter. The UV light sterilizes the filter material by disrupting pathogens or contaminants collected there. A controller of the filter sterilization system may selectively activate individual UV light sources to emit a UV light dose toward the filter material and to achieve a target UV light dose exposure by the expiratory filter. The UV light dose may be selected or determined based on a type of the expiratory filter, a user input, a sterilization mode, or a combination of these. As utilized herein, a UV light dose may refer to a total UV intensity over a total exposure time.

In certain embodiments, the disclosed filter sterilization systems and methods apply continuous or substantially continuous UV light over the course of ventilation of a patient. The present techniques sterilize contaminants retained on or on the expiratory filter, which is substantially fixed in position relative to the UV light, permitting adjustments (e.g., increases or decreases) in exposure time to sterilizing light as well as adjustments (e.g., increases or decreases) in applied UV light intensity. In certain implementations, the intensity of the applied UV light is inversely correlated to an exposure time such that increasing the intensity of the UV light permits a decrease in a total exposure time, and vice versa, to achieve a preset target dose associated with effective or sufficient sterilization as provided herein. One benefit that may be achieved by extending an exposure time, and concurrently decreasing the UV light intensity while maintaining a desired UV light dose, may be reduced UV-associated degradation of the filter material and/or the filter housing. Another benefit may be an increased safety profile associated with a lower intensity UV light setting.

Sterilizing the stationary expiratory filter, rather than the moving expiratory gas flow in the breathing circuit, provides some of these benefits. When UV light shines across the breathing circuit (rather than the filter), contaminants in the expiratory gas cross the UV light as they flow past. The time that any individual moving contaminant particle is exposed to UV light is dictated by the velocity of the gas as it moves through the UV light. As a result, the total UV light dose applied to such expiratory particles can be increased by using higher intensity UV light, but not by increasing a total time that a moving contaminant is exposed to the UV light. Complete and effective sterilization for moving contaminants that experience the UV light only as they cross the light beam may not be possible without using UV light intensities that are damaging to components of the breathing circuit. The sterilization systems described herein operate on the stationary expiratory filter, rather than the moving gas, and thus provide more control over the total UV light dose applied to the expiratory particles. In this manner, the expiratory filter collects active or live pathogens, and the filter sterilizer provided herein disrupts, terminates, kills, or renders inactive any collected active or live pathogens in the expiratory filter. This is in contrast to arrangements that operate directly on the expiratory flow to sterilize moving pathogens that, when killed or inactivated while in the flowing exhalation gases, are collected as dead or inactive pathogens by the expiratory filter.

The sterilized expiratory filters according to the disclosed techniques may be exchanged and/or disposed of with reduced concern for technician exposure to live pathogens and may be handled according to less rigorous protocols as compared to filters that are not sterilized or filters of unknown sterilization status. In addition, the sterilization may permit the expiratory filter to be used over a longer period of time and/or re-used. The filter sterilization system may also permit internal sterilization of internal portions of the ventilator during use. Further, the filter sterilization system may monitor and store sterilization records to improve recordkeeping and compliance with environmental monitoring guidelines for certain types of medical settings.

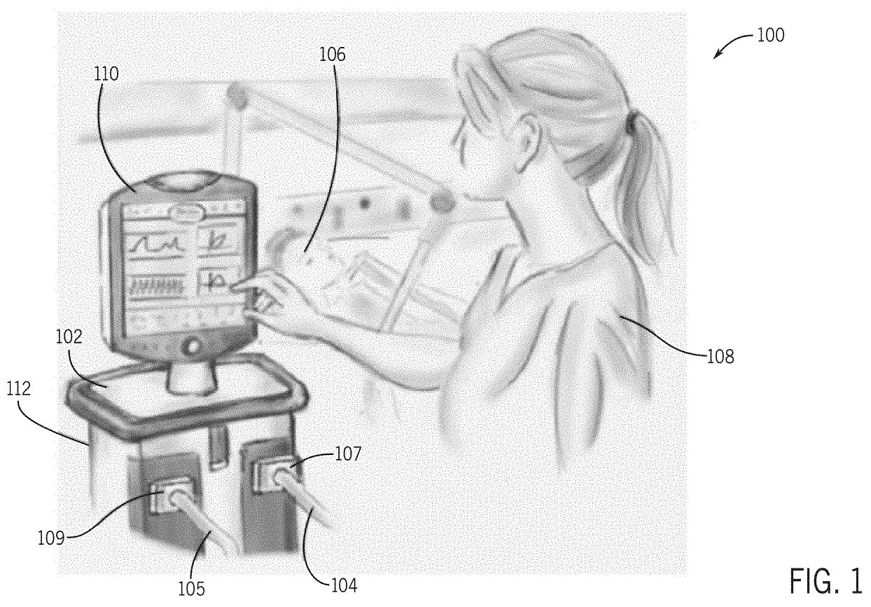

FIG. 1 is a ventilation system 100 that includes a ventilator 102 and a filter sterilization system as disclosed herein. As shown, the ventilation system 100 may include a ventilator 102. The ventilator 102 is used in conjunction with a ventilated patient 106 and by a clinician 108, who interacts with a display 110 of the ventilator 102 The ventilator 102 may engage one or more data collection sensors (not shown) to monitor various parameters that may be measured or calculated based on the closed system between the ventilator 102 and the patient 106. For example, the data collection sensors may collect one or more of gas flow, pressure, volume, or any other data or parameter that may be measured, calculated, or derived based on ventilation of the patient 106, measured at either or both the inhalation port 107 and exhalation port 109 of the ventilator. In an example, the ventilator 102 includes pressure and flow sensors at the inhalation port 107 that measure pressure and flow of the inhalation gases flowing into the inhalation limb 104 of a breathing circuit to the patient 106, and pressure and flow sensors at the exhalation port 109 that measure pressure and flow of the exhalation gases returning through the exhalation limb 105 of the breathing circuit to the ventilator from the patient 106. The ventilator 102 may also receive pressure and flow measurements from sensors along the breathing circuit, but these are optional. This measured, collected, or calculated data may be used by the clinician 108 or ventilator 102 when determining potential adjustments or changes to settings of the ventilator 102 in order to optimize patient-ventilator interaction. The breathing circuit is connected to a non-invasive interface (such as nasal prongs or a nasal, facial, or mouth mask) or an invasive interface (such as an endotracheal tube). The filter sterilization system as provided herein may be coupled to the ventilator 102 and in-line with the exhalation limb 105 at a point before (upstream of) the exhalation port 109, may be coupled after (downstream of) the exhalation port 109, may be integrated on or within a housing 112 of the ventilator 102, and/or may be provided as a modular component that couples to the ventilator 102.

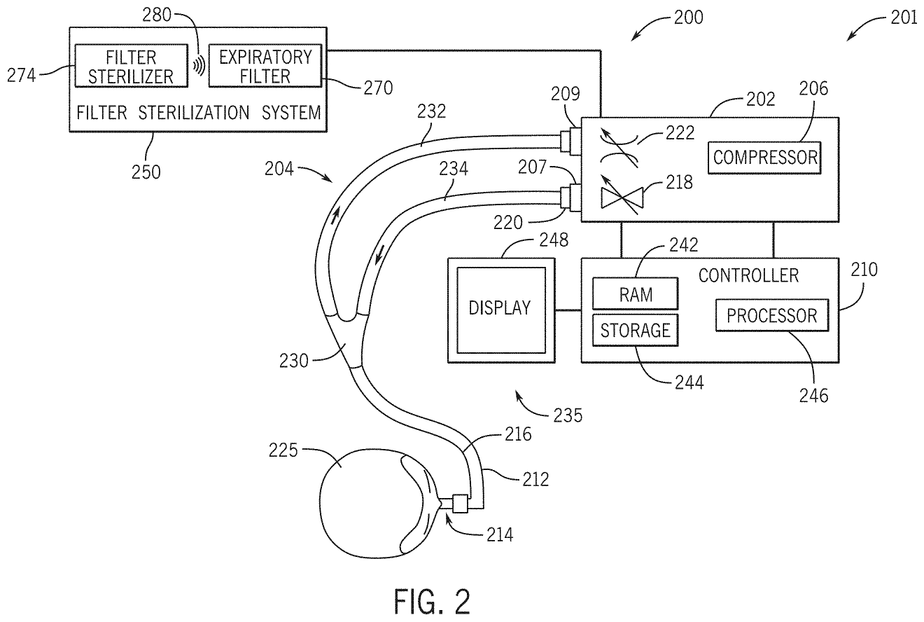

FIG. 2 is a block diagram of a ventilation system 201 that illustrates a ventilator 200 connected to a dual-limb breathing circuit 204 connected to an endotracheal tube 214 which is connected to a human patient 225. The breathing circuit 204 extends from the inhalation port 207 of the ventilator to the endotracheal tube 212, and from there back to the exhalation port 209 of the ventilator 200. The ventilator 200 controls the flow of gases into and out of the patient circuit by controlling (adjusting, opening, or closing) an inhalation flow valve 218 and an exhalation valve 222. Additionally, a humidifier 220 may be placed along the breathing circuit 204 to humidify the inhalation gases to enhance comfort for the patient 225. Pressure and flow sensors are located at the inhalation and exhalation ports 207, 209 to measure parameters of the inhalation and exhalation flows.

The ventilator 200 includes a pneumatic system 202 (also referred to as a pressure generating system 202) for circulating breathing gases to and from patient 225 via the breathing circuit 204 and the endotracheal tube 212. The breathing circuit 204 is a two-limb flexible tube for carrying gases to and from the patient 225. A fitting, typically referred to as a “wye-fitting” 230, connects an inhalation limb 234 and an exhalation limb 232 of the circuit, and couples the circuit to the endotracheal tube 212.

The inhalation limb 234 is connected to the inhalation port 207 and to the inhalation flow valve 218, and the exhalation limb 232 is connected to the exhalation port 209 and the exhalation flow valve 222. A compressor 206 or other source(s) of pressurized gases (e.g., tanks or hoses that supply compressed air, oxygen, and/or helium) provides a gas source for ventilatory support via inhalation limb 234. The pneumatic system 202 may include a variety of other components, including mixing modules, valves, sensors, tubing, accumulators, filters, etc. A controller 210 is operatively coupled with pneumatic system 202, signal measurement and acquisition systems, and an operator interface 235 that may enable an operator to interact with the ventilator 200 (e.g., change ventilator settings, select operational modes, view monitored parameters, etc.). The controller 210 may include hardware memory 242, one or more processors 246, storage 244, and/or other components of the type commonly found in command and control computing devices. In the depicted example, operator interface 220 includes a display 248 that may be touch-sensitive and/or voice-activated, enabling the display 248 to serve both as an input and output device. The ventilation system 201 includes a filter sterilization system 250 that operates to sterilize an expiratory filter 270 and that includes a filter sterilizer 274 that emits sterilizing UV light 280 towards the expiratory filter 270. The filter sterilization system 250 may be configured to integrate with the ventilator 200, and the filter sterilizer 272 may communicate with the controller 210 of the ventilator 200 and may receive provide notification and receive user inputs via the user interface 235. In an embodiment, the filter sterilization system 250 may have a separate controller and user interface.

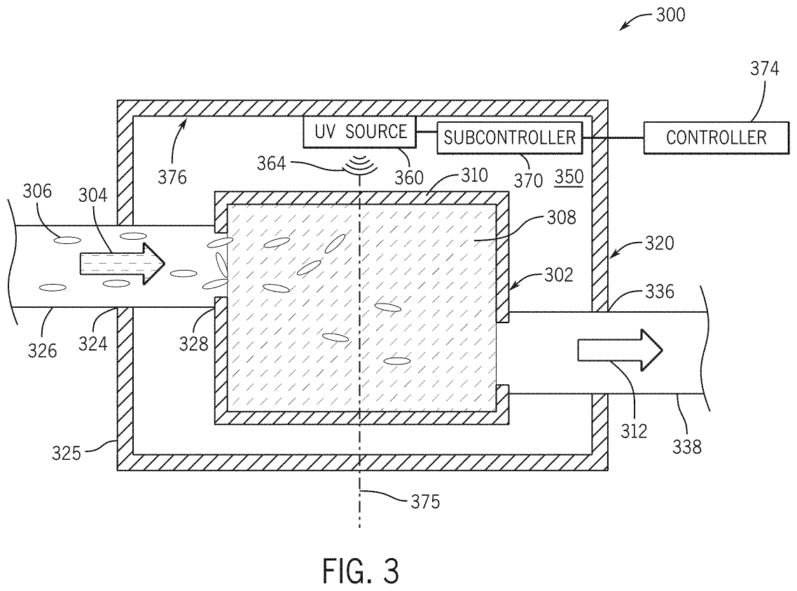

FIG. 3 is a schematic diagram of a filter sterilization system 300. The filter sterilization system 300 sterilizes an expiratory filter 302 positioned along the expiratory gas flow path 304. Exhalation gases in the expiratory gas flow path 304 are directed into the expiratory filter 302, and contaminants 306 (e.g., microorganisms, viruses, small molecules, water droplets) within the exhalation gases may be collected on a filter material 308 that is retained within a filter housing 310 of the expiratory filter 302. A cleansed gas stream 312 is generated and exits from the filter material 308 and out of the expiratory filter 302. As illustrated, the airborne contaminants 306 may therefore accumulate or reside on the filter material 162 and are prevented from exiting in the cleansed gas stream 75.

In the illustrated embodiment, the expiratory filter 302 is coupled to (e.g., positioned within or adjacent to) a filter sterilizer 320. The expiratory filter 302 may be removably coupled to the filter sterilizer 320. Further, the expiratory filter 302 may be removably coupled to the expiratory gas flow path 304. An operator uncouples inlet and outlet ports to uncouple the expiratory filter 302 from the expiratory gas flow path 304. In one example, the filter sterilizer 320 includes one or more sterilizer inlets 324 formed in or on a sterilizer housing 325 to receive a conduit 326 that transfers the exhalation gases into the expiratory filter 302 at a filter inlet 328 and one or more filter outlets 330 that permits exit of gases so that the exhalation gases travel across the filter material 308 while the contaminants 306 are retained on the filter material 308. The exiting cleansed gas stream 312 from the expiratory filter 302 exits the filter sterilizer 320, e.g., through an exit conduit 338. In the illustrated example, the expiratory gas flow path 304, the filter material 308, and the exhaled gas stream 312 are fluidically isolated from an interior space 350 within the filter sterilizer 320 to prevent contaminants 306 from entering the interior space 350. Thus, the filter sterilizer 302 remains relatively clean for an improved serviced life.

The filter sterilizer 320 includes a UV source 360 that emits UV light 364 into the expiratory filter 302 to sterilize the filter material 308, thus sterilizing the expiratory filter 302. In the illustrated embodiment, the UV source 360, e.g., one or more light emitting diodes (LEDs), is coupled to a subcontroller 370 that drives selective activation based on instructions received from a controller 374 (e.g., a controller 210 of the ventilator 200) of the filter sterilization system 300. The subcontroller 370 may include a light drive that operates to change an emitted intensity of the UV light 364 according to instructions from the controller 374. In some cases, the subcontroller 370 includes a processor, memory, and other hardware components similar to those of the controller 374. The filter sterilizer 320 may include a wired or wireless communication component to facilitate communication of the filter sterilizer 320 with other elements of the filter sterilization system 300 or a ventilator (e.g., ventilator 200).

The UV source 360 is coupled to or disposed within a filter-facing surface 376 of the sterilizer housing 325, such that one or multiple individual light sources (e.g., UV-LEDs, UV-C LEDs, sterilizing lamps) of the UV source 360 are arranged to emit the UV light 364 toward at least one surface or location on the filter material 308 of the expiratory filter 302. As illustrated, the UV light 364 is emitted along an axis 375 directly through a filter housing 310 of the expiratory filter 302 into the filter material 308. The UV source 360 may be oriented at any or multiple orientations relative to the filter material 308 to facilitate exposure of the contaminants 306 to sterilizing UV light 364. The UV light may be in a range of 100-400 nm, 100-280 nm (UV-C light), or 207-222 nm (far-UVC light) in an embodiment. In embodiments, the target UV light dose may be between 2,000-13,000 μW·s/cm², based on UV intensity (μW/cm²)×exposure time (seconds). In an embodiment, the UV light dose is selected to be above a minimum threshold that inactivates viruses and other known bacteria such as tuberculosis and legionella.

In contrast to sterilization that may occur while contaminants 306 are in motion and transferred along the gas flow path, the filter sterilization system 300 operates to sterilize the contaminants 306 that are dwelling on or retained within the filter material 308. Sterilization of retained and relatively stationary contaminants 306 permits increased exposure time to sterilizing UV light 364 and more complete and effective sterilization relative to implementations that operate directly on gas transferred within the breathing circuit. For example, a UV light 364 oriented to cross (e.g., orthogonal to) the expiratory gas flow path 304 to sterilize moving contaminants 306 would impinge on an individual contaminant particle 306 for a relatively shorter period of time dictated by the velocity of the expiratory gas flow path 304.

In contrast, the contaminants 306 in the illustrated embodiment are sterilized while dwelling within the filter material 308, permitting a relatively longer UV light exposure than is possible for contaminants 306 moving in and out of range of light directed across the breathing circuit. Because the time exposure to UV light 364 is greater in the filter sterilization system 300 relative to light emitted towards moving contaminants 306, the intensity of the UV light 364 may be tuned to a lower level that is effective for sterilization over the adjustable exposure times available the retained contaminants 306 in the filter material 308 to the UV light 364. That is, both the UV light exposure time and intensity may be changed to set the UV light dose experienced by the retained contaminants 306 in the filter material 308.

Where present, all or a majority of the filter housing 310 of the expiratory filter 302 may be formed of a material that is transmissive or transparent to the UV light 364 to permit penetration of the UV light 364 into the filter material. The sterilizer housing 325, in contrast, may be formed all or in part of a material opaque to UV light 80. For example, the sterilizer housing 325 may be made of a UV opaque plastic, polymer, or metal to reduce leaking of UV light 364 and any unintentional exposure of medical providers to the UV light 364. In embodiments in which the UV source is embedded within a wall of or behind a window formed in the sterilizer housing 325, a portion of the sterilizer housing 325 is transparent to UV light to allow the light to be emitted towards the filter material 308. Further, the sterilizer housing 325 may include internal shielding or UV absorptive elements to further reduce inadvertent light transmission outside of the filter sterilizer 320.

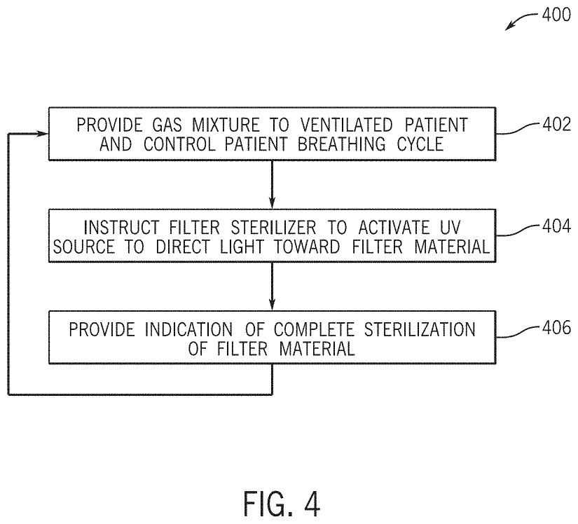

With the above understanding of the components and general operation of the filter sterilization system 300 in mind, further discussion is provided herein regarding certain processes of operating example embodiments of the filter sterilization system, along with certain illustrative user interfaces. For example, FIG. 4 is a flow diagram of a method of operating the filter sterilization system 300, in accordance with some embodiments. The method is generally indicated by reference number 400 and includes various steps or actions represented by blocks. Certain elements of FIG. 4 are discussed with reference to elements illustrated in FIGS. 1-3. It should be noted that the method 400 may be performed as an automated procedure by a system, such as the filter sterilization system 300. Further, certain steps or portions of the method 400 may be performed by separate devices, such as one or more devices illustrated in FIGS. 1-3.

As illustrated, the present embodiment of the method 400 begins with a (e.g., the ventilator 102, the ventilator 200) providing, at step 402, a gas mixture to a ventilated patient. As discussed above with respect to FIGS. 1-2, the ventilator controls delivery of gas to the lungs of the ventilated patient. In one embodiment, the ventilator operates to provide positive pressure ventilation. The ventilated patient exhales into the exhalation stream, which passes through the expiratory filter, where the airborne contaminants are collected for subsequent sterilization.

At step 404, the method includes instructing the filter sterilizer 320 to activate one or more UV sources 360 to direct UV light 364 toward the filter material 308 to provide a target dose of UV light 80 via continuous or selective activation of the one or more UV light sources 360. The system 20 may operate to monitor the applied UV dose and determine if the expiratory filter 70 has been sufficiently sterilized. In an embodiment, at step 404, the system proves an indication of completed sterilization of the filter material 308 of the expiratory filter 302. The completed sterilization may be based on the filter sterilization system 300 tracking the time and intensity of the UV light 364 and determining sterilization has occurred for at least a preset time period and/or a pre-set target total UV dose. However, in other embodiments, no notification is provided, and/or the filter sterilization system 300 does not track completion of sterilization

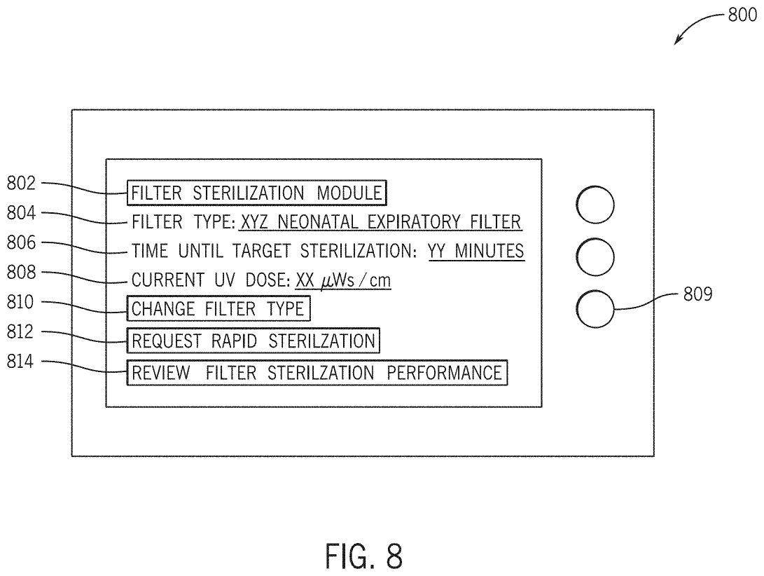

After sterilization, the expiratory filter 302 can then be safely disposed of or exchanged, limiting exposure of medical provider to active pathogens from the patient. The exchange may be performed in conjunction with scheduled maintenance of the filter sterilization system 300. The filter sterilization system 300 may provide a filter exchange notification that is activated based on total ventilator operating time, tracked by the ventilator 200, and that is provided in conjunction with the notification of completed sterilization, and/or the filter exchange notification may include information regarding a filter sterilization status (see FIG. 8).

As part of a maintenance protocol for the filter sterilization system 300, the technician may provide a user input to mark or initiate expiratory filter exchange that causes the filter sterilization system 300 to coordinate completing the sterilization and, in an embodiment, deactivating any active UV sources 360 during the exchange to reduce technician exposure to UV light. The method 400 of FIG. 3 returns to step 402 after receiving a signal triggered by the user input indicative of completion of the exchange of the used expiratory filter 302 for a new expiratory filter 302. The signal indicating completed exchange may permit reactivation of deactivated UV sources 360 to start a new sterilization cycle of the new expiratory filter 302.

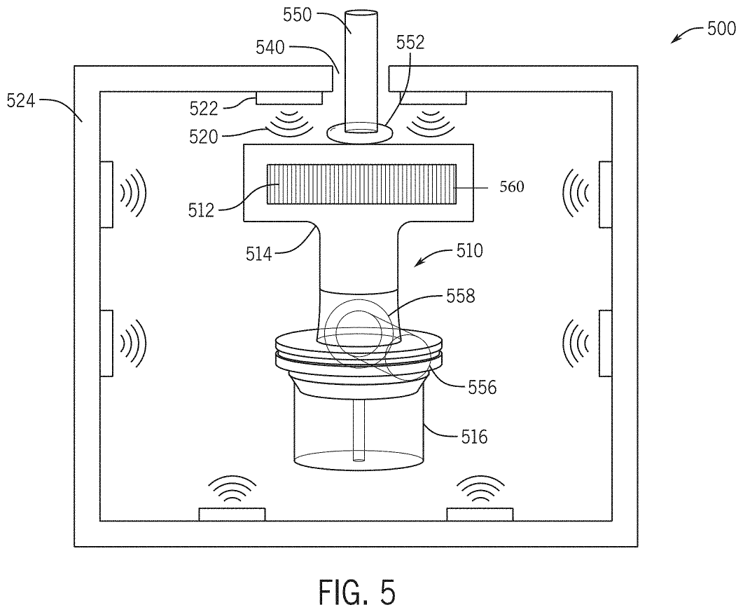

FIG. 5 shows one embodiment of a filter sterilizer 500 disposed around the expiratory filter 510. The expiratory filter 510 includes the filter material 512 within filter housing 514. In this illustrated embodiment, the expiratory filter 510 includes a collection cup 516 for liquids that are in the breathing circuit that enter the expiratory filter 510. The filter sterilizer 500 provides UV light 520 to at least one side or surface of expiratory filter 510. In the depicted embodiment, the UV light 520 can be applied from multiple sides. However, additional arrangements are contemplated. In one example, multiple UV light sources 522 (e.g., UV-C LEDs) are retained on filter-facing surfaces of the housing 524 of the filter sterilizer 30 to provide a direct UV light transmission path into the filter material 512 (and through any UV-transparent filter housing 514). The housing 524 of the filter sterilizer 30 may include one or more reflective surfaces that reflect the emitted UV light 520 into the filter material 512 to enhance sterilization from multiple angles. The UV light sources 522 are individually addressable or controllable by a controller (e.g., the controller 374, see FIG. 3), of the filter sterilizer 500. Accordingly, all or only a subset of the UV light sources 522 can be activated (e.g., turned on) at one time and individually deactivated (e.g., turned off). The UV light sources 522 are individually addressable or addressable in groups in an embodiment to adjust (increase or decrease) an intensity of emitted UV light. In one embodiment, the arrangement of multiple UV light sources 522 serves to provide more complete sterilization by providing additional light transmission paths into the filter material 512 from multiple angles to increase the probability that the emitted UV light 520 impinges the contaminants dwelling on or in the filter material 512 to break down, disrupt, or otherwise render the contaminants sterilized. Further, different UV light sources 522 can be configured to emit at different wavelengths such that the UV light sources 522 provide an array that covers the wavelength range of interest.

In the depicted embodiment, the sterilizer housing 524 is disposed around the expiratory filter 510 in a box or cabinet-type arrangement, which may at least partially surround the expiratory filter 510. In another embodiment, the sterilizer housing 524 may be sized and shaped to be positioned near, on or at least partially around the expiratory filter 510. The sterilizer housing 524 may be sized and shaped to accommodate a variety of types of expiratory filters 510, including special filters and multiple filter arrangements.

The arrangement of the sterilizer housing 524 may include one or more openings 540 to permit entry of exhalation gases to the expiratory filter 510 via an inlet 550 at a filter inlet coupling 552 to the expiratory filter 510 and to permit release of cleansed gases via an outlet 556 that is coupled to the expiratory filter 510 at a filter outlet coupling 558. The filter inlet coupling 552 and/or the filter outlet coupling 558 may include a seal to isolate the exhalation gases from the filter sterilizer 500.

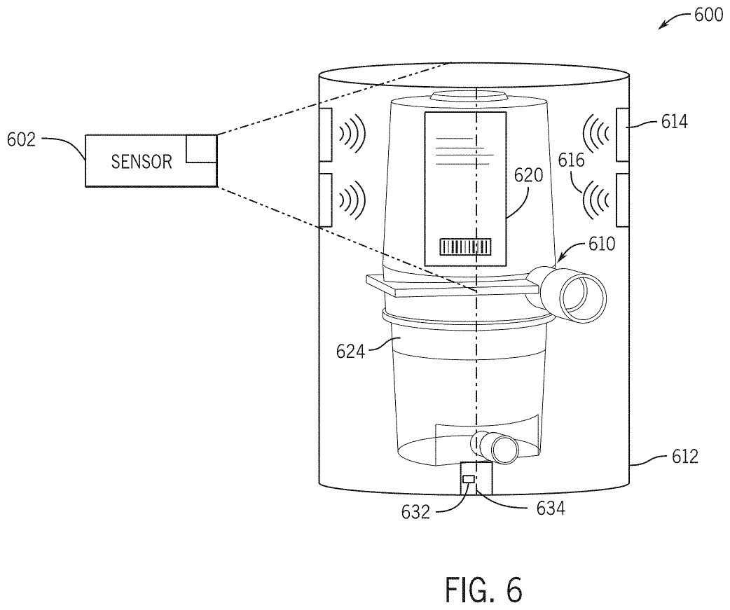

In one embodiment, a user is able to remove and replace the entire expiratory filter 510 from the filter sterilizer 500. In an embodiment, additionally or alternatively, the user is able to exchange filter cartridges 560, formed from or including filter material 512, in and out of the filter housing 514, which remains coupled to the filter sterilizer 500 and facing the sterilizer housing 524. The filter replacement may encompass replacement of the expiratory filter 510 and/or replacement of the filter cartridge 560 with a new cartridge. In embodiments in which the filter sterilizer 500 is automatically turned on and off depending on if the filter sterilizer 500 is loaded, the UV light sources 522 may be activated based on the presence of the expiratory filter 510 and/or the presence of the filter cartridge 560 in the filter housing 514. In one embodiment, the automatic activation and deactivation operates such that the filter sterilizer 500 automatically turns itself on (activates one or more the UV light sources 522) when there is a filter cartridge 560 inside of the filter housing 514, and turns itself off (deactivates one or more the UV light sources 522) when the filter cartridge 560 is removed. In an embodiment, insertion of the filter cartridge 560 into the filter housing 514 generates a signal provided to the filter sterilizer 500 via physical closing a circuit within the filter housing 514 or another type of physical coupling signal, and removal of the filter cartridge stops the signal, indicating removal of the filter cartridge 560 to the filter sterilizer 500. In an embodiment, a presence or absence of the filter cartridge 560 (or the expiratory filter 510) is indicated by a user input to the filter sterilizer 500 or via a sensor signal, as generally discussed with respect to FIG. 6.

FIG. 6 shows an arrangement of a filter sterilizer 600 than includes a sensor 602 that senses a presence of the expiratory filter 610 in the sterilizer housing 612. The filter sterilization system may be programmed to permit activation of the UV light sources 614 to emit UV light 616 only upon receipt of the sensor signal indicative of the presence of the filter 610 inside the housing 612. For example, the sensor 602 may include an optical emitter that emits a light beam across the space of the sterilizer housing 612 and a detector positioned to detect the light beam. When the expiratory filter 610 is not present, the light beam impinges the detector at an intensity that is close to an emitted light intensity. When the expiratory filter is present, the signal at the detector is reduced in a characteristic manner, and the sensor 602 provides a signal indicative of the expiratory filter 610 being in place. The signal may be used to permit activation of the one or more UV light sources 614.

In another embodiment, the sensor 602 may include a camera or RFID reader to detect or identify filter information 620 as well as a presence of the expiratory filter 610. In the depicted example, the filter information 620 is provided or printed on the filter housing 624. The filter information may be provided in a QR code, bar code, or other format that is read or acquired by the sensor 602 and provided to the system. For example, the sensor 602 may acquire image data of the filter information 620, and send the image data to a controller (e.g., the controller 374, see FIG. 3). The controller may extract the information in the printed text using text recognition or information in a code using pattern matching techniques. Once extracted, the information is used to identify or determine a filter type of the expiratory filter 610 and access a matched set of sterilization parameters associated with the filter type. The filter information 620 may include a type of filter, a size of the filter, and a manufacturer of the filter. In one example, the sterilization parameters are used to select a subset of UV light sources that are active and that are set based on a configuration and position of the expiratory filter 610 within the sterilizer housing 612.

In the illustrated embodiment, the sterilizer housing 612 of the filter sterilizer 600 is arranged as a two-part receiving chamber. The depicted arrangement may be provided as a separate or standalone unit that may be used to retrofit a ventilator that does not include an internal expiratory filter coupling or integral filter sterilizer. During application of the UV dose and while the UV light source 614 is actively emitting UV light 616, the filter sterilizer 600 can activate a lock 632 on the access door 634 to prevent inadvertent exposure of caregivers to UV light.

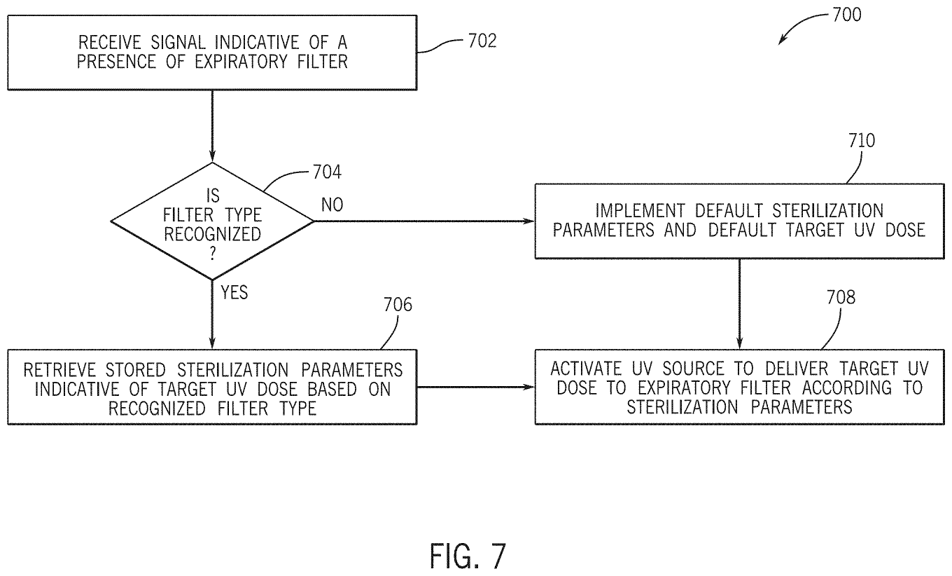

FIG. 7 is a flow diagram of a method of determining sterilization parameters for an expiratory filter, in accordance with some embodiments. The method is generally indicated by reference number 700 and includes various steps or actions represented by blocks. Certain steps of the method 700 are discussed in the context of elements referenced in FIGS. 1-3 and 5-6. Further, certain steps or portions of the method 700 may be performed by separate devices, such as one or more devices illustrated in FIGS. 1-2.

At step 702, the method 700 receives a signal indicative of a presence of an expiratory filter (e.g., expiratory filter 610, FIG. 6). The signal can be a sensor signal, e.g., from the sensor 602 of FIG. 6, that also captures filter information. In one example, the sensor signal is from a camera that captures an image of filter information. The sensor signal includes image data from which the filter information is extracted, e.g., using text recognition. The extracted information may include a manufacturer name, a filter number, filter media, filter efficiency, filter housing material, etc. The extracted information is used to search a stored set of recognized filter types of the system. For recognized filters, the method 700 retrieves stored sterilization parameters that includes target dose information based on the filter type at step 706. Such information may also include activation instructions to activate one or more UV light sources of the filter sterilizer at step 708. In one example, the UV lights sources may be activated based on the configuration, size, or shape of the expiratory filter to optimally direct the UV light to the filter material. If the filter material is oriented towards a top half of the filter sterilizer, the stored sterilization parameters may include instructions to activate a subset of the UV light sources positioned in the top half. The sterilization parameters may include a target dose based on the total size of the expiratory filter and/or the UV spectrum transmissivity of the filter's housing material. For filters that are not recognized by the system, the system can activate default sterilization parameters at step 710.

FIG. 8 shows an example user interface 800 to provide user input to control sterilization parameters. The user interface may be included on a ventilator (see FIGS. 1-2). The user interface 800 includes a menu, illustrated here as a filter sterilization module 802 that may display filter information, such as a filter type indication 804, a countdown or time remaining indication 806, current UV light dose indication 808, and so forth. User input options may include manual entry of filter type, adjusting a filter sterilization UV dose, or based on log in credentials, such that higher ranked users can perform further tasks with filter sterilization system than lower ranked users.

The user interface 800 can also provide information to controller by user selectable buttons 809 or a touch screen input, such as a change filter type input 810, rapid sterilization request input 812, or review performance input 814.

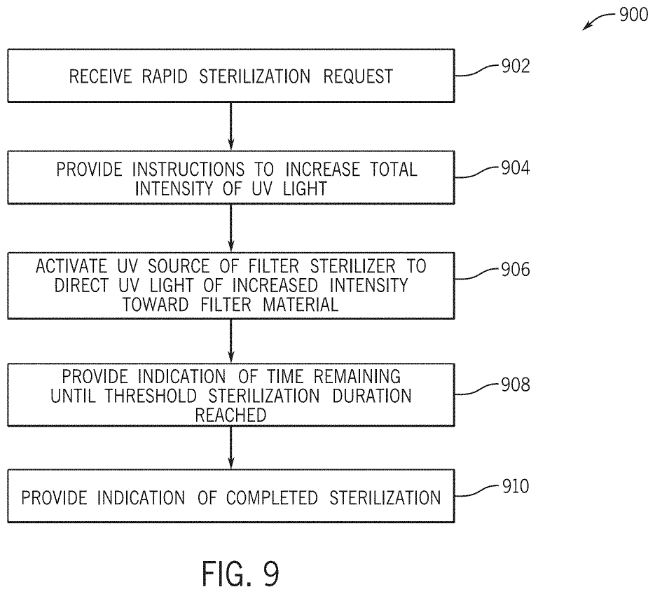

FIG. 9 is a flow diagram of a method 90 of rapid sterilization of the expiratory filter, in accordance with some embodiments. Certain steps of the method 900 are discussed in the context of elements referenced in FIGS. 1-3, 5-6, and 8. Further, certain steps or portions of the method 900 may be performed by separate devices, such as one or more devices illustrated in FIGS. 1-2.

The method 900 initiates with receiving a rapid sterilization request 902, e.g., via the request rapid sterilization input 812, illustrated in FIG. 8. In an embodiment, a technician may wish to shorten the time remaining until sterilization is complete (e.g., as shown on the user interface 800 via the time remaining indication 806), and can activate a rapid sterilization mode. In one example, a technician may wish to complete maintenance on the ventilator during their shift. However, the expiratory filter may be inaccessible during sterilization. Activation of rapid sterilization causes the filter sterilizer to provide instructions to increase the intensity of the UV light from the one or more UV light sources at step 904, e.g., by increasing the driving voltage via the light drive. The UV light sources are activated to emit UV light having the increased intensity toward the filter material. Sterilization progress is tracked on the user interface 800 on the time remaining indication 806 at step 908, and the system provides an indication of completed sterilization when the rapid sterilization is complete at step 910. In one embodiment, the completion of sterilization permits access to the expiratory filter by unlocking an access door of the filter sterilizer that is locked during sterilization. A rapid sterilization or rapid access input may cause a final bolus of UV light to be applied before the access door is unlocked to permit filter exchange.

The system may track the UV light dose by monitoring an activation time and a light intensity emitted from each active UV light source to determine if the filter material has received sufficient UV light to provide the indication of completed sterilization. The sterilization dose or target dose may be set as a threshold total UV light exposure, and may be stored as a sterilization parameter, e.g., a filter-specific sterilization parameter or a default sterilization parameter. In the case of rapid sterilization, the increase in emitted intensity causes the UV light dose associated with completed sterilization to be achieved more quickly.

As provided herein, the filter sterilization systems and methods apply a sterilizing dose of UV light to an expiratory filter to facilitate release of cleansed respiratory gases into the ambient environment and safe filter exchange and disposal. The filter sterilization systems and methods may be used during operation of the ventilator, e.g., simultaneously with ventilation and pathogen exposure, to provide more efficient filter sterilization.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A system, comprising:
a ventilator comprising a housing having an expiratory port, and an expiratory filter located downstream of the expiratory port, wherein the expiratory filter comprises filter material that collects pathogens from expiratory gases received at the expiratory port; and
a filter sterilizer coupled to the expiratory filter, wherein the filter sterilizer comprises an ultraviolet (UV) light source oriented at the filter material.

2. The system of claim 1, comprising a controller that operates to activate the UV light source.

3. The system of claim 1, wherein the UV light source comprises a plurality of UV light emitting diodes (LEDs), and wherein the UV light comprises a wavelength in a range of 100-400 nanometers.

4. The system of claim 1, wherein the UV light source comprises a plurality of UV light sources, and wherein the system comprises a controller that operates to individually activate each UV light source based on an identified or determined type of the expiratory filter.

5. The system of claim 1, wherein the filter material of the expiratory filter is fluidically isolated from the filter sterilizer.

6. The system of claim 1, wherein the expiratory filter comprises a filter inlet to receive the exhalation gases, wherein the exhalation gases comprise the pathogens and wherein the expiratory filter releases cleansed gases from a filter outlet to enter an ambient environment, and wherein a level of pathogens in the cleansed gases is reduced relative to the exhalation gases.

7. The system of claim 1, wherein the ventilator comprises a controller that operates to:
provide a gas mixture to the ventilated patient; and
instruct the UV light source to emit a target dose of UV light oriented toward the filter material of the expiratory filter.

8. The system of claim 7, wherein the controller provides an indication of completed sterilization after the target dose of UV light is applied.

9. The system of claim 1, wherein the filter sterilizer comprises an access door and a lock that is activated to lock the access door while the UV light source emits UV light.

10. A filter sterilizer, comprising:
a housing sized and shaped to accommodate an expiratory filter;
a plurality of ultraviolet (UV) light sources coupled to the housing; and
a controller communicatively coupled to the plurality of UV light sources, wherein the controller:
receives an indication of a presence of the expiratory filter in the housing; and
activates at least one UV light source of the plurality of UV light sources based on the indication.

11. The filter sterilizer of claim 10, wherein the filter sterilizer is disposed within a ventilator housing of a ventilator and wherein the controller is a controller of the ventilator.

12. The filter sterilizer of claim 10, wherein controller activates the at least one UV light source by providing activation instructions to a light drive of the at least one UV light source.

13. The filter sterilizer of claim 10, wherein the controller individually addresses light sources of the plurality of light sources to activate only a subset of the plurality of UV light sources.

14. The filter sterilizer of claim 13, wherein the activated subset is selected based on a size or type of the expiratory filter.

15. The filter sterilizer of claim 10, wherein the housing of the filter sterilizer is opaque to UV light.

16. The filter sterilizer of claim 10, wherein the controller receives an indication of removal of the expiratory filter from the housing and deactivates the at least one UV light source based on the indication of removal.

17. The filter sterilizer of claim 10, comprising an optical sensor coupled to the housing, wherein the optical sensor generates sensor data comprising the indication of the presence of the expiratory filter in the housing.

18. The filter sterilizer of claim 10, comprising a camera that acquires image data of the expiratory filter and provides the image data to the controller, and wherein the controller identifies a size or type of the expiratory filter based on the image data.

19. The filter sterilizer of claim 18, wherein the controller adjusts activation of the at least one UV light source based on the identified size or type of the expiratory filter.

20. A system, comprising:
an expiratory filter that receives exhalation gases from an expiratory limb of a breathing circuit of a ventilated patient, wherein the expiratory filter comprises filter material that collects contaminants present in the exhalation gases; and
an ultraviolet (UV) light source oriented at the expiratory filter; and
a controller communicatively coupled to the UV light source, wherein the controller:
activates the UV light source to emit a UV light dose to the filter material of the expiratory filter; and
generates a signal indicative of completed sterilization in response to the UV light dose being emitted.

21. The system of claim 20, wherein the filter material is disposed within a filter housing that is transparent to UV light generated by the UV light source.

22. The system of claim 20, wherein the UV light source emits UV-C light.

23. The system of claim 20, wherein the UV light dose is a threshold UV intensity over time delivered to the filter material of the expiratory filter.

24. The system of claim 20, wherein the controller:
receives a signal indicative of requested access to the expiratory filter; and
provides an indication of remaining time until completed sterilization.

25. The system of claim 20, wherein the controller:
receives a user input for rapid access to the expiratory filter or rapid sterilization; and
increases a UV intensity of the UV light source in response to the user input.

26. The system of claim 20, wherein the controller:
receives an input indicative of a particular type of the expiratory filter and sets the UV light dose based on the input.

27. The system of claim 20, wherein the controller is coupled to an access door and locks the access door until the signal indicative of completed sterilization is generated.

28. The system of claim 20, wherein the controller receives a signal indicative of requested sterilization of the expiratory filter.
